(19) United States
(12) Patent Application Publication
(10) Pub. No.: US 2024/0358632 A1
(43) Pub. Date: Oct. 31, 2024

(54) READY TO USE COMPOSITIONS OF CETRORELIX ACETATE

(71) Applicants: Ravishankar KOVI, North Brunswick, NJ (US); Raghu Rami Reddy Kasu, Pearl River, NY (US); Thupalli Ajey Kumar REDDY, Bangalore, Karnataka (IN); Yekkanti VAMSHI, Bangalore, Karnataka (IN); Jayraman KANNAPPAN, Vadodara, Gujrat (IN); Kamlesh CHAUAHAN, Vadodara, Gujrat (IN)

(72) Inventors: Ravishankar KOVI, North Brunswick, NJ (US); Raghu Rami Reddy Kasu, Pearl River, NY (US); Thupalli Ajey Kumar REDDY, Bangalore, Karnataka (IN); Yekkanti VAMSHI, Bangalore, Karnataka (IN); Jayraman KANNAPPAN, Vadodara, Gujrat (IN); Kamlesh CHAUAHAN, Vadodara, Gujrat (IN)

(73) Assignee: RK Pharma Inc., Pearl River, NY (US)

(21) Appl. No.: 18/682,785

(22) PCT Filed: May 26, 2022
(86) PCT No.: PCT/IB2022/054963

§ 371 Date: Feb. 9, 2024

(51) Int. Cl. A61K 9/00; A61K 9/08; A61K 38/09; A61K 47/26

(52) U.S. Cl. CPC A61K 9/0019; A61K 9/08; A61K 38/09; A61K 47/26

(57) ABSTRACT

The present application discloses a ready-to-use liquid parenteral pharmaceutical composition, that has Cetrorelix or Cetrorelix acetate. The composition also has Cetrolix or its pharmaceutically acceptable salt, ethanol, and water for injection, and a pH adjusting agent. The composition may also contain pharmaceutically acceptable excipients. The Cetrolix or Cetrolix acetate preparation can have a pH lower than 7.

### RELATED APPLICATION

This application is a PCT International application and claims priority to and the benefit of U.S. Provisional Patent Application Ser. No. 63/231,822 filed on Aug. 11, 2021, the contents of which are incorporated herein in its entirety.

### FIELD OF THE INVENTION

The present invention provides a ready-to-use solutions of Cetrorelix or its pharmaceutically acceptable salt or any combination thereof. The present invention also provides a manufacturing methods of ready-to-use solutions of Cetrorelix. The present invention also provides a parenteral dosage form with a ready-to-use aqueous solution of Cetrorelix acetate avoiding the need of reconstitution.

### BACKGROUND OF THE INVENTION

Chemically, Cetrorelix, synthetic decapeptide is gonadotropin releasing hormone antagonist (GnRH antagonist) acetyl-D-3-(2′-naphtyl)-alanine-D-4-chlorophenylalanine-D-3-(3′-pyridyl)-alanine-L-serine-L-tyrosine-D-citruline-L-leucine-L-arginine-L-proline-D-alanine-amide (C₇₀H₉₂ClN₁₇O₁₄) having the following formula:

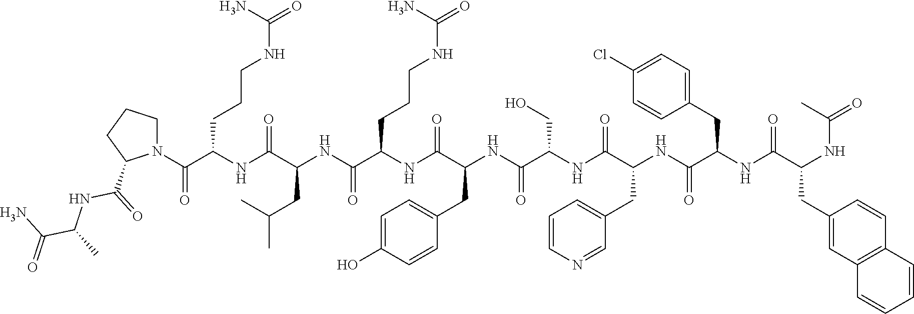 [SMILES: CC(=O)N[C@H](Cc1ccc2ccccc2c1)C(=O)N[C@H](Cc1ccc(Cl)cc1)C(=O)N[C@H](Cc1cccnc1)C(=O)N[C@@H](CO)C(=O)N[C@@H](Cc1ccc(O)cc1)C(=O)N[C@H](CCCNC(N)=O)C(=O)N[C@@H](CC(C)C)C(=O)N[C@@H](CCCNC(N)=O)C(=O)N1CCC[C@H]1C(=O)N[C@H](C)C(N)=O]

Marketed Cetrotide® (cetrorelix acetate for injection) 0.25 mg or 3 mg is a sterile lyophilized powder intended for subcutaneous injection after reconstitution with Sterile Water for Injection, (pH 5-8), that comes supplied in either a 1.0 mL (for 0.25 mg vial) or 3.0 mL (for 3 mg vial) pre-filled syringe. Each vial of Cetrotide® 0.25 mg (multiple dose regimen) contains 0.26-0.27 mg cetrorelix acetate, equivalent to 0.25 mg cetrorelix, and 54.80 mg mannitol. Each vial of Cetrotide® 3 mg (single dose regimen) contains 3.12-3.24 mg cetrorelix acetate, equivalent to 3 mg cetrorelix, and 164.40 mg mannitol.

Marketed formulation Cetrotide® is a lyophilized powder intended for subcutaneous injection after reconstitution. Assuming the lyophilization and a lyophilizate was prepared to avoid peptide instability problems, there still an unmet need to address the problems associated with the cost, time cycle and affordability to the patients and patient compliance.

However, our impugned invention has brought a solution to the problem with the drawbacks associated with Cetrotide®—(1) high manufacturing cost along with tedious time consuming lyophilization process; (2) product is not convenient because it is not ready-to-use and it has complexity of requiring reconstitution before administration; and (3) reconstituted solution is stable only for a short period of time. Cetrotide® lyophilized product thus did not fulfil a need for a ready-to-use aqueous sterile pharmaceutical formulation.

Cetrotide® (cetrorelix acetate for injection) is indicated for the inhibition of premature LH surges in women undergoing controlled ovarian stimulation.

Cetrorelix as a compound was disclosed in U.S. Pat. No. 4,800,191 patent.

U.S. Pat. No. 7,718,599 disclosed the pharmaceutical compositions suitable for parenteral administration comprising peptides in the form of acid salts like acetate, gluconate, glucuronate, lactate, citrate, ascorbate, benzoate or phosphate salts in dissolved or dispersed form and at least one of the acid selected from a group of gluconic acid, glucaric acid or galactouronic acid for forming the salts in free acid form. The pharmaceutically acceptable acids are capable of imparting a pH in between 2.5 to 4.5 to the composition which helps in suppressing aggregation of Cetrorelix acetate. Further, the US '599 patent also discloses lyophilized aggregation-free Cetrorelix preparation containing various cyclodextrins. However, U.S. Pat. No. 7,718,599 did not report the effect of pH on the chemical stability of Cetrorelix. Our impugned invention overcomes the prior art by not containing the acid selected from a group of gluconic acid, glucaric acid or galactouronic acid, also not containing cyclodextrins, and additionally our impugned invention provides a stability of the composition up to 9 months along with the controlling the impurities within the ICH criteria.

US 2013/0303464 patent application discloses a ready-to-use aqueous preparation of Cetrorelix comprising Cetrorelix acetate, glacial acetic acid, a tonicity adjusting agent and water for injection. A suitable pH was illustrated by working examples where the pH was about 3. The preferred pH according to the invention was pH 2.8 to 3.5. Our impugned invention differs from the invention disclosed in the US' 464 application and it relates to a composition comprising a solvent, preferably ethanol, optionally mannitol and a pH adjusting agent which can maintain the pH lower than 7.

U.S. Pat. No. 7,214,662 disclosed aqueous solutions of peptides including Cetrorelix acetate and suggested solutions to the problem of aggregation. It taught that carboxylic acids and especially hydroxycarboxylic acids, preferably gluconic acid in combination with a surfactant improves solubility thereby reduces aggregation with the help of surfactants. The use of carboxylic acid according to U.S. Pat. No. 7,214,662 resulted in a low pH such as pH 2.5 to 3. Our disclosed invention does not propose the utilization of surfactants to reduce the problem of aggregation.

WO2016059592A1 discloses non aqueous system for ready-to-use injectable peptide drug. Our impugned invention differs from the invention disclosed in WO' 592 in such a way that it doesn't contain the polyethylene glycol, propylene glycol and an antioxidant.

WO2020254952 A1 discloses a stable ready-to-use solution using cyclodextrin and glacial acetic acid and a surfactant; wherein the pH of the said formulation is from about 3.5 to 8.5. Our impugned invention differs from the invention disclosed in WO' 952 in such a way that it doesn't contain cyclodextrin and a surfactant.

US20210121517 patent application discloses stable aqueous ready-to-inject sterile solution comprising lactic acid, which results in pH range of 3.5 to 5 and an osmotic agent, whereas our proposed invention provides a composition and manufacturing method wherein it does not contain lactic acid and cyclodextrin.

Moreover, the prescribing information guides that Cetrotide® should be reconstituted before use. To avoid such a drawback and provide an economical formulation, we have found stable parenteral solution containing Cetrorelix acetate in infusion bag and vial, which is stable more than 3 months at room temperature.

The currently marketed form of Cetrorelix for injection is costly to manufacture and inconvenient to use because it is not in a ready-to-use format. Hence there is an unmet need in the market to make the compositions which are readily administrable and more patient compliant. Additionally, our inventors of the present invention contemplate, and surprisingly found an aqueous and ready-to-use Cetrorelix acetate solution compositions without any specialty excipients likewise disclosed in the prior arts and having acceptable bioavailability is highly desirable. In particular, the ready-to-use, injectable formulations described herein are stable, economical, allow medical personals to use prepared containers containing an injectable formulation off the shelf without additional preparation, avoid potential contamination problems, and eliminate dosage errors.

### SUMMARY OF THE INVENTION

An embodiment of present invention provides a ready-to-use injectable Cetrorelix acetate solution and manufacturing methods of the same.

Another embodiment of present invention provides a ready-to-use injectable Cetrorelix acetate solution which is easy to administer without need of any reconstitution step and has a improve solubility, stability, and safety profile.

In one or more embodiments there is provided a parenteral formulation of Cetrorelix acetate. Formulation prepared by this application are stored in vial, prefilled syringe, auto injector, or infusion bag.

In another embodiment of the present invention provides the ready to use pharmaceutical formulation of Cetrorelix or its pharmaceutically acceptable salts, any co-crystals or any combination thereof can be prepared by using solvents, polyols, with optionally other pharmaceutically acceptable excipients. Specifically, the present invention provides a ready-to-use liquid injectable parenteral pharmaceutical composition, comprising Cetrorelix and a first excipient, wherein the first excipient is one or more selected from the group consisting of solubilizers like polyols but not limited to alkyl or aryl alcohol, sugar alcohols, diluted alcohols, ethanol, isopropyl alcohol, mannitol, sorbitol, xylitol, erythritol, lactitol, isomaltose and a combination thereof or mixtures thereof. Preferably, in one of the compositions, the first excipient according to the present invention can be selected from the group consisting of solvents, such as one or more of ethanol, isopropyl alcohol. The first excipient can be preferably ethanol in one of the compositions of the present invention.

In another embodiment, the composition of the present invention may also contain the first excipient selected from the mannitol, sucrose, lactose or combinations thereof.

In a preferred embodiment, the composition of the present invention wherein, the first excipient's role is to improve the solubility of the active ingredient Cetrorelix acetate or may act as stabiliser and may also act an isotonicity agent or may also act as diluent. The first excipient preferably may be ethanol or mannitol or sucrose or lactose. The second excipient may be used to make up the volume up to to 1 ml and further the sodium hydroxide or hydrochloric acid was used to adjust the pH lower than 7. preferably pH lower than 5, more preferably pH lower than 3.

In a preferred embodiment, the composition of the present invention also comprises a second excipient that mainly plays a stabilizing role. The second excipient can be selected from the group consisting of pH adjusting agents like acids and bases but not limited to sodium hydroxide, hydrochloric acid, citric acid, acetic acid, phosphoric acid, succinic acid, potassium hydroxide, ammonium hydroxide, etc. and combinations thereof or any mixtures thereof.

In one of the other embodiments of the present invention Cetrorelix formulations are also prepared without using first excipient in formulation.

In still further embodiments provided are ready-to-use liquid parenteral formulations including Cetrorelix acetate, one or more pharmaceutically acceptable stabilizing agents and solvents, co-solvents, and/or solubilising agent, pH adjusting agents, preservative, tonicity adjusting agent.

In one of the other embodiments of the present invention provide the storage-stable, ready-to-use, injectable compositions as GnRH antagonist.

In one of the other embodiments of the present invention provides a stable ready to use compositions of Cetrorelix or Cetrorelix acetate for at least 6 months at room temperature, at least 8 months at cool temperature and for at least 3 months at accelerated conditions.

In one of the other embodiments of the present invention provides a stable ready to use compositions of Cetrorelix or Cetrorelix acetate wherein the impurities are controlled within the acceptable ICH guidelines, preferably Cetrorelix acid impurity lower than 1%, Desalanine Cetrorelix acid lower than 1%, Nona Cetrorelix acid lower than 1%, Hexa Cetrorelix impurity lower than 2%, Hepta Cetrorelix impurity lower than 2%, and Nal Cetrorelix acid impurity lower than 1%.

The details of one or more embodiments of the invention are set forth in the description below. Other features, objects and advantages of the invention will be apparent from the description.

### DETAILED DESCRIPTION OF THE PREFERRED EMBODIMENTS

The present invention now will be described more fully hereinafter with reference to the accompanying examples and experiments, in which illustrative embodiments of the invention are mentioned. The examples provided herein are exemplary and not limiting the scope of the invention; and any modification or variation can be apparent to any person skilled in the art. This invention may, however, be embodied in many different forms and should not be construed as limited to the embodiments set forth herein; rather, these embodiments and examples are provided so that this disclosure will be thorough and complete, and will fully convey the scope of the invention to those skilled in the art.

The terminology used herein is for the purpose of describing particular embodiments only and is not intended to be limiting the scope of the invention. As used herein, the singular forms “a”, “an” and “the” are intended to include the plural forms as well, unless the context clearly indicates otherwise. Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

As used herein, “Cetrorelix” refers to Cetrorelix and the pharmaceutically acceptable salts, solvates, hydrates, crystals and anhydrous forms and any pro-drugs thereof.

As used here in “ready-to-use” when used in connection with a Cetrorelix formulation refers to a liquid pharmaceutical formulation that includes Cetrorelix acetate in dissolved or solubilized form and/or is intended to be used as such or upon further dilution in intravenous, subcutaneous, intramuscular and/or parenteral route diluents.

As used herein, the term “sterile” refers to unless otherwise specified, liquid composition sterilized conventionally or by in-process sterilization.

As used herein, the term “parenteral” refers to, route of administration of the disclosed invention, which is subcutaneous, intramuscular, intravenous, intraperitoneal, intrathecal, intra-articular, unless otherwise particularly specified.

As used herein, and unless otherwise specified, the term “storage-stable” refers to physical and chemical stability of the liquid formulation disclosed in the invention. Liquid Cetrorelix acetate-containing composition or formulation having sufficient physical and chemical stability to allow storage in its package at a convenient suitable temperature above the freezing point of the composition or formulation for a commercially reasonable period of time.

The phrase “physical stability” of the formulation refers to maintenance of appearance, colour or colourless state, dissolved oxygen level, head space oxygen level and particulate matter and the term “chemical stability” provides to formation of drug-related impurities in terms of total impurities, single maximum individual impurity, or maximum individual impurities, known or unknown. For pharmaceutical products, stability is required for commercially relevant times after manufacturing, such as for about 6, 12, 18, 24, or 36 months, during which time a product is kept in its original packaging under specified storage conditions.

As used herein, and unless otherwise specified, the term “about” means an acceptable error for a particular value as determined by one of ordinary skill in the art, which depends in part on how the value is measured or determined. In certain embodiments, the term about means within 10%, 9%, 8%, 7%, 6%, 5%, 4%, 3%, 2%, 1%, 0.5%, 0.1%, or may be in range up to 0.001%.

The aqueous parenteral dosage form comprising the ready-to-use sterile pharmaceutical formulation of Cetrorelix according to the present invention contains Cetrorelix acetate or any pharmaceutically acceptable salt thereof, at a concentration ranging from 0.26 mg/ml to 0.28 mg/ml, which amount is equivalent to 0.25 mg/ml of Cetrorelix base. Preferably, Cetrorelix acetate is present in the ready-to-use sterile aqueous pharmaceutical solution at a concentration equivalent to 0.25 mg/ml of Cetrorelix base.

In one embodiment, the parenteral dosage form comprising the ready-to-inject sterile, stable aqueous solution of Cetrorelix according to the present invention comprises a pH adjusting agent at a concentration sufficient to adjust the pH lower than 7.0

In one embodiment, the parenteral dosage form comprising the ready-to-inject sterile, stable aqueous solution of Cetrorelix according to the present invention comprises a pH adjusting agent at a concentration sufficient to adjust the pH preferably at 3-7.

In one embodiment, the present invention provides a ready-to-use liquid injectable parenteral pharmaceutical composition comprising Cetrorelix and a first excipient, wherein the first excipient is one or more selected from the group consisting of solubilizers, polyols and a combination thereof.

Preferably the first excipient according to the present invention can be selected from the group comprising of solvents, such as one or more of but not limited to alkyl or aryl alcohol, sugar alcohols, diluted alcohols, ethanol, isopropyl alcohol, mannitol, sorbitol, xylitol, erythritol, lactitol, isomaltose, sucrose, lactose, or mixtures thereof, which are well known in the art. The first excipient can be preferably ethanol.

Preferably the first excipient according to the present invention can be selected from the group comprising of solvents, such as one or more of but not limited to alkyl or aryl alcohol, sugar alcohols, diluted alcohols, ethanol, isopropyl alcohol, mannitol, sorbitol, xylitol, erythritol, lactitol, isomaltose, sucrose, lactose, or mixtures thereof, which are well known in the art. The first excipient can be preferably mannitol or sucrose or lactose.

More preferably, the first excipient according to the present invention will be functioning as diluent, stabiliser, isotonicity agent, solvent, solubiliser, osmotic agent or combinations thereof.

In another embodiment, the present invention provides a ready-to-use liquid injectable parenteral pharmaceutical composition comprising Cetrorelix acetate and second excipient. Second excipient mainly plays a role in pH adjusting process, preferably controlling the pH lower than 7, more preferably lower than 5.

In one of the other embodiment of the present invention Cetrorelix formulations are also prepared without using first excipient in formulation.

The second excipient can be selected from the group of pH adjusting agents comprising of acids and bases but not limited to sodium hydroxide, hydrochloric acid, acetic acid, citric acid, phosphoric acid, succinic acid, potassium hydroxide, ammonium hydroxide, etc. and combinations or mixtures thereof.

Pharmaceutically acceptable additional excipients or adjuvants optionally include but are not limited to one or more preservatives, polymers, buffers, surfactants, chelating agents, dispersing agents, binding agents, tonicity modifying agents and antioxidants.

Examples of pharmaceutically acceptable pH adjusting agents include but are not limited to sodium hydroxide, hydrochloric acid, citric acid, acetic acid, phosphoric acid, succinic acid, potassium hydroxide, ammonium hydroxide, etc. and combinations thereof. These pH adjusting agents helps to maintain the pH lower than 7, preferably lower than 5, more preferably lower than 4.

Pharmaceutically acceptable vehicles comprise but are not limited to 0.9% NaCl, sterile water for injection, 5% of dextrose, lactated ringer solution and combinations thereof.

The parenteral dosage form according to the present invention may be in the form of clear injectable solution, suspension or emulsion.

The storage-stable, ready-to-use injectable Cetrorelix acetate-containing formulations disclosed herein do not require any additional reconstitution step at the time of administration.

Formulations in accordance with the present disclosure have a controlled impurity profile suitable for regulatory approval at various storage conditions. The storage-stable ready-to-use Cetrorelix acetate formulations may be stored at about 0° C. to about 25° C. The storage-stable, ready-to-use Cetrorelix acetate formulations for injection may retain at least 90% of the potency of Cetrorelix acetate after storage for at least nine months at about 0° C. to about 25° C. temperature and 60% relative humidity, preferably at least six months at about 0° C. to about 25° C. temperature and 60% relative humidity, more preferably at least three months at about 0° C. to about 25° C. temperature and 60% relative humidity.

The storage-stable, ready-to-use, injectable formulations may be formulated to provide single or multiple dosage administration. The single dosage formulation may be packaged in an ampoule, a vial, or a syringe or an infusion bag or a prefilled syringe. Multiple dosage formulations may be packaged in a vial. Multiple dosage formulations may preferably include at least one preservative.

The formulations have a pH value from about 2 to about 10. In some embodiments the pH range is from about 3 to about 8, preferable lower than 3.

Storage-stable ready-to-use, injectable liquid formulations disclosed herein contain Cetrorelix acetate having a purity in the range of about 97% to about 102%. In some embodiments the formulation contains Cetrorelix acetate having a purity of from about 90% to about 110%, preferably more than 95%, and more preferably less than 105%. In some embodiments the formulation contains Cetrorelix acetate having a purity of about 100%.

The formulation comprises excipients such as solvents, such as one or more of but not limited to alkyl or aryl alcohol, sugar alcohols, diluted alcohols, ethanol, isopropyl alcohol, mannitol, sorbitol, xylitol, erythritol, lactitol, isomaltose or mixtures thereof, which are well known in the art. The concentration of mannitol used in the ready to use formulation of cetrorelix acetate is in the range of 0-70 mg/ml, more preferably about 40-60 mg/ml.

Formulations in accordance with the present disclosure contain solvents like ethanol. The concentration of ethanol used in the ready to use formulation of cetrorelix acetate is in the range of 25-75% v/v, more preferably about 30-70% v/v

Water for injection (WFI) optionally may be used to make up the desired volume as a vehicle for the formulation about to 2 ml, preferably about 1 ml, more preferably about 0.5-1 ml.

Certain specific aspects and embodiments of the present application will be explained in more detail with reference to the following examples, without wishing to be bound by a theory, which are provided only for purposes of illustration and should not be construed as limiting the scope of the present application in any manner.

### EXAMPLES

The following examples are for the illustration only and are not intended in any way to limit the scope of the present invention.

### Example 1

TABLE 1

| Ingredients | Mg/vial | % w/v |
|---|---|---|
| Cetrorelix Acetate IH | 0.26-0.27 mg | 0.026 to 0.027 |
| Water for Injection USP | Q.S | 1 ml |
| Sodium Hydroxide USP-NF | Q.S | Q.S |
| Hydrochloric Acid USP-NF | Q.S | Q.S |

### Preparation:

Required quantity of Cetrorelix acetate was dissolved in sterile water for injection. pH was adjusted to lower than 7 using sodium hydroxide or/and hydrochloric acid, as required. Volume was made up to 1 ml with sterile water for injection. The vials were sealed and stored. Stability data is summarized in Table 1A

TABLE 1A

| | | 25° C. /60% RH | | 5° C. ± 3° C. | | | |
|---|---|---|---|---|---|---|---|
| Time point | Initial | 1.5 M | 2.5 M | 1.5 M | 2.5 M | 3.5 M | 4.5 M |
| % Purity | 99.54 | 99.28 | 98.91 | 99.87 | 99.64 | 99.8 | 99.8 |

| | |
|---|---|
| pH | 3-7 |

Related Substances

| | | | | | | | |
|---|---|---|---|---|---|---|---|
| Tetra-cetrorelix acid | ND | ND | ND | ND | ND | ND | ND |
| Desalanine Cetrorelix acid | 0.08 | 0.05 | 0.08 | ND | ND | 0.03 | 0.04 |
| Cetrorelix acid | 0.07 | 0.21 | 0.53 | 0.02 | 0.06 | 0.12 | 0.1 |
| Nona Cetrorelix acid | ND | ND | ND | ND | ND | ND | ND |
| Nal Cetrorelix acid | ND | ND | ND | ND | ND | ND | ND |
| Hexa Cetrorelix | ND | ND | ND | ND | ND | ND | ND |
| Hepta Cetrorelix | ND | ND | ND | ND | ND | ND | ND |
| Highest Unspecified Impurity | 0.14 | 0.18 | 0.21 | 0.09 | 0.17 | 0.05 | 0.05 |
| Total Impurities | 0.46 | 0.72 | 1.08 | 0.13 | 0.36 | 0.2 | 0.2 |

### Example 2

TABLE 2

| Ingredients | Mg/vial | % w/v |
|---|---|---|
| Cetrorelix Acetate IH | 0.26-0.27 mg | 0.026 to 0.027 |
| Ethyl alcohol USP | Q.S | 0.5 ml |
| Water for Injection USP | Q.S | 0.5 ml |
| Sodium Hydroxide USP-NF | Q.S | Q.S |
| Hydrochloric Acid USP-NF | Q.S | Q.S |

### Preparation:

Required quantity of Cetrorelix acetate was dissolved in ethanol and sterile water for injection in a ratio (1:1 to 1:9) was added. pH was adjusted to lower than 7 using sodium hydroxide or/and hydrochloric acid, as required. Volume was made up to 1 ml with sterile water for injection. The vials were sealed and stored. Stability data is summarized in Table 2A.

TABLE 2A

| | Composition | | | | | | | | | | |
|---|---|---|---|---|---|---|---|---|---|---|---|
| | WFI + Ethanol + Cetrorelix | | | | | | | | | | |
| | pH | | | | | | | | | | |
| | 3-7 | | | | | | | | | | |
| | | 25° C. /60% RH Upright | | | | 5° C. ± 3° C. Upright | | | | 40 C./75% Upright | |
| Time point | Initial | 1 M | 2 M | 3 M | 6 M | 1 M | 2 M | 3 M | 6 M | 1 M | 3 M |
| % Purity | 99.7 | 100.3 | 99.9 | 99.9 | 100 | 99.9 | 99.5 | 99.4 | 99.2 | 99.99 | 99.4 |

Related Substance

| | | | | | | | | | | | |
|---|---|---|---|---|---|---|---|---|---|---|---|
| Tetra-cetrorelix acid | ND | ND | ND | ND | ND | ND | ND | ND | ND | ND | ND |
| Des-alanine Cetrorelix acid | 0.04 | 0.05 | 0.06 | 0.03 | 0.02 | 0.06 | 0.05 | 0.04 | 0.02 | 0.04 | 0.05 |
| Cetrorelix acid | 0.01 | ND | 0.02 | 0.02 | 0.08 | ND | 0.01 | 0.01 | ND | 0.10 | 0.22 |
| Nona Cetrorelix acid | ND | ND | ND | 0.02 | 0.05 | ND | ND | ND | ND | ND | 0.25 |
| Nal Cetrorelix acid | ND | ND | ND | 0.02 | 0.02 | ND | ND | ND | ND | 0.06 | 0.31 |
| Hexa Cetrorelix | ND | ND | ND | ND | ND | ND | ND | ND | ND | ND | ND |
| Hepta Cetrorelix | ND | ND | ND | ND | 0.02 | ND | ND | ND | ND | 0.03 | 0.1 |
| Highest Unspecified Impurity | 0.11 | 0.11 | 0.15 | 0.16 | 0.16 | 0.12 | 0.13 | 0.11 | 0.09 | 0.31 | 0.74 |
| Total Impurities | 0.27 | 0.42 | 0.53 | 0.59 | 0.86 | 0.48 | 0.43 | 0.36 | 0.21 | 1.03 | 4.32 |

### Example 3

TABLE 3

| Ingredients | Mg/viaL | % w/v |
|---|---|---|
| Cetrorelix Acetate IH | 0.26-0.27 mg | 0.026 to 0.027 |
| Mannitol USP | 54.8 | 5.48 |
| Ethyl alcohol USP | Q.S | 0.5 ml |
| Water for Injection USP | Q.S | 0.5 ml |
| Sodium Hydroxide USP-NF | Q.S | Q.S |
| Hydrochloric Acid USP-NF | Q.S | Q.S |

### Preparation:

Required quantity of Cetrorelix acetate, mannitol and ethanol was dissolved in sterile water for injection. pH was adjusted to lower than 7 using sodium hydroxide or/and hydrochloric acid, as required. Volume was made up to 1 ml with sterile water for injection. The vials were sealed and stored. Stability data is summarized in Table 3A

TABLE 3A

pH
3-7

25° C./60% RH

| Time point | Initial | 1.5 M | 2.5 M |
|---|---|---|---|
| % Purity | 99.64 | 99.32 | 99.55 |

Related Substances

| | | | |
|---|---|---|---|
| Tetra-cetrorelix acid | ND | ND | ND |
| Desalanine Cetrorelix acid | 0.08 | 0.09 | 0.05 |
| Cetrorelix acid | 0.06 | 0.1 | 0.07 |
| Nona Cetrorelix acid | ND | ND | ND |
| Nal Cetrorelix acid | ND | 0.06 | ND |
| Hexa Cetrorelix | ND | ND | ND |
| Hepta Cetrorelix | ND | ND | ND |
| Highest Unspecified Impurity | 0.14 | 0.17 | 0.18 |
| Total Impurities | 0.26 | 0.68 | 0.45 |

### Example 4

TABLE 4

| | | |
|---|---|---|
| Ingredients | Mg/vial | % w/v |

| | | | |
|---|---|---|---|
| Cetrorelix Acetate IH | 0.26-0.27 | mg | 0.026 to 0.027 |
| Dextrose USP | 50 | mg | 5% |

| | | |
|---|---|---|
| Water for injection | Q.S | 1 ml |
| Sodium Hydroxide USP-NF | Q.S | Q.S |
| Hydrochloric Acid USP-NF | Q.S | Q.S |

### Preparation:

Dissolved required quantity dextrose in water for injection and add the Cetrorelix acetate to the solution, the pH was adjusted to lower than 7 using sodium hydroxide or/and hydrochloric acid, as required. Volume was made up to 1 ml with sterile water for injection. The vials were sealed and stored. Stability data is summarized in Table 4A.

TABLE 4A

| | pH | | | | | |
|---|---|---|---|---|---|---|
| | 3-7 | | | | | |
| | | 25° C. /60% RH | | | 5° C. ± 3° C. | |
| Time point | Initial | 1 M | 2 M | 3 M | 1 M | 3 M |
| % Purity | 99.69 | 98.93 | 98.63 | 95.78 | 99.7 | 98.97 |

Related Substances

| | | | | | | |
|---|---|---|---|---|---|---|
| Tetra-cetrorelix acid | ND | ND | ND | ND | ND | ND |
| Desalanine Cetrorelix acid | 0.06 | ND | 0.07 | 0.1 | 0.05 | 0.05 |
| Cetrorelix acid | 0.01 | 0.17 | 0.09 | 0.38 | 0.02 | 0.11 |
| Nona Cetrorelix acid | ND | ND | ND | ND | ND | ND |
| Nal Cetrorelix acid | ND | ND | ND | ND | ND | ND |
| Hexa Cetrorelix | ND | 0.38 | 0.81 | 1.66 | ND | ND |
| Hepta Cetrorelix | ND | ND | ND | ND | ND | 0.28 |
| Highest Unspecified | 0.11 | 0.13 | 0.11 | 1.52 | 0.1 | 0.1 |
| Impurity | | | | | | |
| Total Impurities | 0.26 | 0.98 | 2.3 | 4.05 | 0.26 | 0.91 |

### Example 5

TABLE 5

| Ingredients | Mg/viaL | % w/v |
|---|---|---|
| Cetrorelix Acetate IH | 0.26-0.27 mg | 0.026 to 0.027 |
| Mannitol USP | 54.8 | 5.48 |
| Water for Injection USP | Q.S | 1 ml |
| Sodium Hydroxide USP-NF | Q.S | Q.S |
| Hydrochloric Acid USP-NF | Q.S | Q.S |

### Preparation:

Required quantity of Cetrorelix acetate and mannitol was dissolved in sterile water for injection. pH was adjusted to lower than 7 using sodium hydroxide and/or further adjusted with hydrochloric acid, as required. Volume was made up to 1 ml with sterile water for injection. The vials were sealed and stored. Stability data is summarized in Table 5A

TABLE 5A

| Time point | | 1 M | 2 M | 3 M | 4 M | 9 M | 1 M | 2 M |
|---|---|---|---|---|---|---|---|---|
| Condition | Initial | 5 ± 3° C. | | | | | 25° C./60% | |
| pH | 3-7 | | | | | | | |
| % Assay | 99.62 | 99.8 | 99.57 | 99.57 | 99.57 | 98.3 | 99.67 | 99.06 |

Related substances

| | | | | | | | | |
|---|---|---|---|---|---|---|---|---|
| Tetra-cetrorelix acid | ND | ND | ND | ND | ND | ND | ND | ND |
| Desalanine Cetrorelix acid | 0.05 | 0.05 | 0.06 | 0.06 | 0.06 | 0.09 | 0.06 | 0.06 |
| Cetrorelix acid | 0.08 | 0.08 | 0.12 | 0.14 | 0.09 | 0.24 | 0.08 | 0.47 |
| Nona Cetrorelix acid | ND | ND | ND | ND | ND | ND | ND | ND |
| Nal Cetrorelix acid | ND | ND | ND | ND | ND | ND | ND | ND |
| Hexa Cetrorelix | ND | ND | ND | ND | ND | ND | ND | ND |
| Hepta Cetrorelix | ND | ND | ND | ND | ND | ND | ND | ND |
| Highest Unspecified Impurity | 0.12 | 0.04 | 0.12 | 0.09 | 0.07 | 0.14 | 0.09 | 0.12 |
| Total Impurities | 0.38 | 0.2 | 0.43 | 0.43 | 0.43 | 1.03 | 0.33 | 0.96 |

### Example 6

TABLE 6

| Ingredients | Mg/mL | % w/v |
|---|---|---|
| Cetrorelix Acetate IH | 0.26-0.27 mg | 0.026 to 0.027 |
| Mannitol USP | 27.4 | 2.74 |
| Water for Injection USP | Q.S to 1 mL | 1 ml |
| Sodium Hydroxide USP-NF | Q.S | Q.S |
| Hydrochloric Acid USP-NF | Q.S | Q.S |

### Preparation:

Required quantity of Cetrorelix acetate and mannitol was dissolved in sterile water for injection. pH was adjusted to lower than 7 using sodium hydroxide or hydrochloric acid, as required. Volume was made up to 1 ml with sterile water for injection. The vials were sealed and stored. Stability data is summarized in Table 6A.

TABLE 6A

| | pH | | | |
|---|---|---|---|---|
| | 3-7 | | | |
| | | 5° C. ± | 25° C./ | 40° C./ |
| | | 3° C. | 60% RH | 75% RH |
| Time point | Initial | 1 M | 1 M | 1 M |
| % Purity | 99.63 | 99.8 | 99.65 | 98.00 |

Related Substances

| | | | | |
|---|---|---|---|---|
| Tetra-cetrorelix acid | ND | ND | ND | ND |
| Desalanine Cetrorelix acid | 0.05 | 0.05 | 0.05 | 0.08 |
| Cetrorelix acid | 0.07 | 0.07 | 0.08 | 0.16 |
| Nona Cetrorelix acid | ND | ND | ND | ND |
| Nal Cetrorelix acid | ND | ND | ND | ND |
| Hexa Cetrorelix | ND | ND | ND | ND |
| Hepta Cetrorelix | ND | ND | ND | ND |
| Highest Unspecified Impurity | 0.11 | 0.04 | 0.08 | 1.56 |
| Total Impurities | 0.37 | 0.2 | 0.35 | 2.00 |

### Example 7

TABLE 7

| | | |
|---|---|---|
| Ingredients | Mg/mL | % w/v |

| | | | |
|---|---|---|---|
| Cetrorelix Acetate IH | 0.26-0.27 | mg | 0.026 to 0.027 |
| Mannitol USP | 54.80 | mg | 5.48 |

| | | |
|---|---|---|
| Glacial acetic acid USP | Q.S | Q.S |
| Water for Injection USP | Q.S to 1 mL | 1 ml |

### Preparation:

Required quantity of Cetrorelix acetate and mannitol was dissolved in sterile water for injection. pH was adjusted to lower than 7 using glacial acetic acid. Volume was made up to 1 ml with sterile water for injection. The vials were sealed and stored. Stability data is summarized in Table 7A.

TABLE 7A

pH
3-7

5° C. ± 3° C.

| Time point | Initial | 1 M | 2 M |
|---|---|---|---|
| % Purity | 99.80 | 99.70 | 99.40 |

Related Substances

| | | | |
|---|---|---|---|
| Tetra-cetrorelix acid | ND | ND | ND |
| Desalanine Cetrorelix acid | 0.03 | 0.03 | 0.05 |
| Cetrorelix acid | ND | 0.03 | 0.04 |
| Nona Cetrorelix acid | ND | ND | ND |
| Nal Cetrorelix acid | ND | ND | ND |
| Hexa Cetrorelix | ND | ND | ND |
| Hepta Cetrorelix | ND | ND | ND |
| Highest Unspecified Impurity | 0.04 | 0.07 | 0.08 |
| Total Impurities | 0.10 | 0.12 | 0.17 |

### Example 8

TABLE 8

| | | |
|---|---|---|
| Ingredients | Mg/mL | % w/v |

| | | | |
|---|---|---|---|
| Cetrorelix Acetate IH | 0.26-0.27 | mg | 0.026 to 0.027 |
| Sucrose USP | 50-100 | mg | 5 to 10% |

| | | |
|---|---|---|
| Glacial acetic acid USP | Q.S | Q.S |
| Water for Injection USP | Q.S to 1 mL | 1 ml |

### Preparation:

Required quantity of Cetrorelix acetate and sucrose was dissolved in sterile water for injection. pH was adjusted to lower than 7 using glacial acetic acid. Volume was made up to 1 ml with sterile water for injection. The vials were sealed and stored.

### Example 9

TABLE 9

| | | |
|---|---|---|
| Ingredients | Mg/mL | % w/v |

| | | | |
|---|---|---|---|
| Cetrorelix Acetate IH | 0.26-0.27 | mg | 0.026 to 0.027 |
| Lactose USP | 50-100 | mg | 5 to 10% |

| | | |
|---|---|---|
| Glacial acetic acid USP | Q.S | Q.S |
| Water for Injection USP | Q.S to 1 mL | 1 ml |

### Preparation:

Required quantity of Cetrorelix acetate and lactose was dissolved in sterile water for injection. pH was adjusted to lower than 7 using glacial acetic acid. Volume was made up to 1 ml with sterile water for injection. The vials were sealed and stored.

### Commercial Advantages and Superior Aspects of the Present Invention Over the Prior Arts/Marketed Preparation:

| | Impurity | | Excipients | |
|---|---|---|---|---|
| Preparations | limit | Dosage form | Solvents used | Purity |
| Marketed | 1.5% | Lyophilised | Mannitol | lower |
| preparation | | cake | | |
| Present | 1.0% | Ready to use | Ethanol/Water | higher |
| invention | | solution | | |
| Present | 1.0% | Ready to use | Mannitol/ethanol | higher |
| invention | | solution | | |

| | | Overall | Lyophilization/ |
|---|---|---|---|
| Preparations | Yield | processing time | Drying time |
| Prior arts | 99% | Upto 5 hrs | 36-48 hrs |
| Present invention | 99% | 1 hr to 2 hr | No lyophilization process |

Without wishing to be bound to a theory, the compositions, and methods of making the compositions described in the present invention is believed to be an improved over the compositions disclosed in the prior arts in the terms of stability at cool temperature, room temperature and accelerated temperature, which is commercially scalable, economical, and provides a ready to use formulations by reducing the reconstitution time.

While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth hereinabove but rather that the claims be construed as encompassing all the features of patentable novelty which reside in the present invention, including all features which would be treated as equivalents thereof by those skilled in the art to which the invention pertains.

1. A ready-to-use liquid pharmaceutical preparation of Cetrorelix or Cetrorelix acetate for parenteral administration, comprising:
a. Cetrorelix or its pharmaceutically acceptable salt in a range of 0.01-0.1% w/v,
b. Ethanol,
c. Water for Injection and a pH adjusting agent, and optionally other pharmaceutically acceptable excipients.

2. The pharmaceutical preparation as claimed in claim 1, wherein the Cetrorelix or Cetrorelix acetate preparation has a pH lower than 7.

3. A ready-to-use liquid pharmaceutical preparation of Cetrorelix or Cetrorelix acetate for parenteral administration, comprising:
a. Cetrorelix 0.01-0.1% w/v,
b. Mannitol,
c. Water for Injection, a pH adjusting agent, and a pH lower than 7.

4. A ready-to-use liquid pharmaceutical preparation of Cetrorelix or Cetrorelix acetate for parenteral administration, comprising:
a. Cetrorelix 0.01-0.1% w/v,
b. Sucrose,
c. Water for Injection, a pH adjusting agent, and a pH lower than 7.

5. A ready-to-use liquid pharmaceutical preparation of Cetrorelix or Cetrorelix acetate for parenteral administration, comprising:
a. Cetrorelix 0.01-0.1% w/v,
b. Lactose,
c. Water for Injection, a pH adjusting agent, a pH lower than 7.

6. A ready-to-use liquid pharmaceutical preparation of Cetrorelix or Cetrorelix acetate for parenteral administration as claimed in claim 1, wherein the preparation is stable for a period of more than 5 months at 25° C./60% RH (relative humidity).

7. A ready-to-use liquid pharmaceutical preparation of Cetrorelix or Cetrorelix acetate for parenteral administration as claimed in claim 1, wherein the preparation is stable for a period of more than 5 months at 5° C.±3° C.

8. A ready-to-use liquid pharmaceutical preparation of Cetrorelix or Cetrorelix acetate for parenteral administration as claimed in claim 1, wherein the preparation is stable for a period of more than 3 months at 40° C./75% RH (relative humidity).

9. A ready-to-use liquid pharmaceutical preparation of Cetrorelix or Cetrorelix acetate for parenteral administration, comprising:
Cetrorelix acid lower than 1%,
Desalanine Cetrorelix acid lower than 1%,
Nona Cetrorelix acid lower than 1%,
Hexa Cetrorelix lower than 2%,
Hepta Cetrorelix lower than 2%, and
Nal Cetrorelix acid lower than 1%.

10. A pharmaceutical composition of Cetrorelix or Cetrorelix acetate for parenteral administration, comprising:
Cetrorelix acid lower than 1%,
Desalanine Cetrorelix acid lower than 1%,
Nona Cetrorelix acid lower than 1%,
Hexa Cetrorelix lower than 2%,
Hepta Cetrorelix lower than 2%, and
Nal Cetrorelix acid lower than 1%.
